(19) United States
(12) Patent Application Publication
(10) Pub. No.: US 2025/0143288 A1
(43) Pub. Date: May 8, 2025

(54) METHOD OF USING PLASMALOGEN CONTAINED IN PROCESSED ASCIDIAN

(71) Applicant: SUN CHLORELLA CORP., Kyoto (JP)

(72) Inventors: Osamu MURAKAMI, Osaka (JP); Teruki HIRAMI, Osaka (JP); Hideo TAKEKOSHI, Kyoto (JP); Masaki FUJISHIMA, Kyoto (JP); Masakuni SANDAIJI, Kyoto (JP)

(21) Appl. No.: 18/632,460

(22) Filed: Apr. 11, 2024

(30) Foreign Application Priority Data
Nov. 6, 2023 (JP) 2023-189693

(51) Int. Cl. A01N 1/02

(52) U.S. Cl. CPC A01N 1/125

(57) ABSTRACT

Method of using a plasmalogen contained in a processed ascidian: freeze-drying a Halocynthia roretzi without encystment to obtain a processed ascidian; storing the processed ascidian at room temperature to 35 degrees C. for two months or more after the freeze drying; and using the processed ascidian stored for two months or more as described above to use a plasmalogen contained in the processed ascidian.

### BACKGROUND OF THE INVENTION

### 1. Field of the Invention

The present invention relates to a method of using a plasmalogen contained in a processed ascidian, a method of producing a processed Halocynthia roretzi or another processed ascidian, and a processed ascidian.

### 2. Description of Related Art

JP 2016-210696 A discloses a learning memory ability enhancer used for a healthy mammal, the learning memory ability enhancer including a plasmalogen, in which 90% or more of the plasmalogen is ethanolamine plasmalogen and choline plasmalogen, the plasmalogen is extracted from an avian tissue, and preferably, a mass ratio of ethanolamine plasmalogen:choline plasmalogen contained in the plasmalogen is 1:5 to 5:1.

In addition, JP 2019-162042 A discloses an invention relating to, as an extract from fish mates or aquatic animals, a plasmalogen-type high-phospholipid-content extract containing plasmalogen-type phospholipid with high purity and having a reduced arsenic content, and a method of producing the plasmalogen-type high-phospholipid-content extract.

### SUMMARY OF THE INVENTION

The present invention relates to a method of using a plasmalogen contained in a processed ascidian, a method of producing a processed ascidian having good temporal stability of the contained plasmalogen, a processed ascidian, and a processed ascidian having improved temporal stability of the contained plasmalogen.

A method of using a plasmalogen contained in a processed ascidian of the present invention comprises the steps of: freeze-drying a Halocynthia roretzi or another ascidian without encystment to obtain a processed ascidian; storing the processed ascidian at room temperature to 35 degrees C. for two months or more after the freeze drying; and using the processed ascidian stored for two months or more as described above to use a plasmalogen contained in the processed ascidian. According to this method, the plasmalogen contained in the processed ascidian can be effectively used even after the processed ascidian is stored at room temperature to 35 degrees C. for two months or more.

The method of producing a processed ascidian of the present invention is to produce a processed ascidian having good temporal stability of contained plasmalogen by freeze-drying a Halocynthia roretzi or another ascidian without encystment.

The processed ascidian of the present invention is a processed product of a Halocynthia roretzi or another ascidian without encystment, and the temporal stability of the contained plasmalogen is improved by freeze drying.

The processed ascidian of the present invention for using contained plasmalogen is a processed ascidian obtained by freeze-drying a Halocynthia roretzi or another ascidian without encystment, and is used for using the plasmalogen contained in the processed ascidian after storage at room temperature to 35 degrees C. for two months or more after the freeze drying.

### BRIEF DESCRIPTION OF THE DRAWING

FIG. 1 is a graph showing a change over time in a content of plasmalogen in 100 g of a processed ascidian of an example.

FIG. 2 is a graph showing the change over time in the content of plasmalogen in 100 g of an ascidian extract powder of Comparative Example.

### DETAILED DESCRIPTION OF THE INVENTION

A method of using a plasmalogen contained in a processed ascidian of the present invention, a method of producing a processed ascidian, a processed ascidian, and a processed ascidian for using a contained plasmalogen can be described, for example, as follows.

1. A method of using a plasmalogen contained in a processed ascidian comprising the steps of: freeze-drying a Halocynthia roretzi or another ascidian without encystment to obtain a processed ascidian; storing the processed ascidian at room temperature to 35 degrees C. for two months or more after the freeze drying; and using the processed ascidian stored for two months or more as described above to use a plasmalogen contained in the processed ascidian.

2. The method according to the above 1, further comprising a step of subjecting the processed ascidian to use of a plasmalogen contained in the processed ascidian.

3. The method according to the above 1 or 2, further comprising a step of treating the Halocynthia roretzi or another ascidian without encystment with hot water before performing the freeze drying.

4. The method according to any one of the above 1 to 3, wherein a plasmalogen content in a predetermined mass of the processed ascidian after freeze drying is 10% or more, 30% or more, or, 50% or more of the plasmalogen content in the predetermined mass immediately after freeze drying after a lapse of two months at room temperature.

5. The method according to any one of the above 1 to 3, wherein a plasmalogen content in a predetermined mass of the processed ascidian after freeze drying is 30% or more of the plasmalogen content in the predetermined mass immediately after freeze drying after a lapse of two months in an environment of 25 degrees C.

6. The method according to any one of the above 1 to 3, wherein a plasmalogen content in a predetermined mass of the processed ascidian after freeze drying is 10% or more, or, 30% or more of the plasmalogen content in the predetermined mass immediately after freeze drying after a lapse of two months in an environment of 35 degrees C.

7. The method according to any one of the above 1 to 6, wherein a substance other than water is not substantially added to the Halocynthia roretzi or another ascidian.

8. A method of using a plasmalogen contained in a processed ascidian comprising the steps of: freeze-drying a Halocynthia roretzi or another ascidian without encystment to obtain a processed ascidian; storing the processed ascidian at room temperature to 35 degrees C. for six months or more after the freeze drying; and using the processed ascidian stored for four months or more as described above to use a plasmalogen contained in the processed ascidian.

9. The method according to the above 8, further comprising a step of subjecting the processed ascidian to use of a plasmalogen contained in the processed ascidian.

10. The method according to the above 8 or 9, further comprising a step of treating the Halocynthia roretzi or another ascidian without encystment with hot water before performing the freeze drying.

11. The method according to any one of the above 8 to 10, wherein a plasmalogen content in a predetermined mass of the processed ascidian after freeze drying is 10% or more, 20% or more, or, 40% or more of the plasmalogen content in the predetermined mass immediately after freeze drying after a lapse of six months at room temperature.

12. The method according to any one of the above 8 to 10, wherein a plasmalogen content in a predetermined mass of the processed ascidian after freeze drying is 20% or more of the plasmalogen content in the predetermined mass immediately after freeze drying after a lapse of six months in an environment of 25 degrees C.

13. The method according to any one of the above 8 to 10, wherein a plasmalogen content in a predetermined mass of the processed ascidian after freeze drying is 10% or more, or, 30% or more of the plasmalogen content in the predetermined mass immediately after freeze drying after a lapse of six months in an environment of 35 degrees C.

14. The method according to any one of the above 8 to 13, wherein a substance other than water is not substantially added to the Halocynthia roretzi or another ascidian.

15. A method of producing a processed ascidian having good temporal stability of contained plasmalogen, the method of producing a processed ascidian comprising freeze-drying a Halocynthia roretzi or another ascidian without encystment.

16. The production method according to the above 15, further comprising treating the Halocynthia roretzi or another ascidian without encystment with hot water before performing the freeze drying.

17. The production method according to the above 15 or 16, wherein a substance other than water is not substantially added to the Halocynthia roretzi or another ascidian.

18. The production method according to any one of the above 15 to 17, wherein a plasmalogen content in a predetermined mass of the processed ascidian after freeze drying is 30% or more of the plasmalogen content in the predetermined mass immediately after freeze drying after a lapse of two months in an environment of 25 degrees C.

19. The production method according to any one of the above 15 to 17, wherein a plasmalogen content in a predetermined mass of the processed ascidian after freeze drying is 10% or more, or, 30% or more of the plasmalogen content in the predetermined mass immediately after freeze drying after a lapse of two months in an environment of 35 degrees C.

20. The production method according to any one of the above 15 to 17, wherein a plasmalogen content in a predetermined mass of the processed ascidian after freeze drying is 20% or more of the plasmalogen content in the predetermined mass immediately after freeze drying after a lapse of six months in an environment of 25 degrees C.

21. The production method according to any one of the above 15 to 17, wherein a plasmalogen content in a predetermined mass of the processed ascidian after freeze drying is 10% or more, or, 30% or more of the plasmalogen content in the predetermined mass immediately after freeze drying after a lapse of six months in an environment of 35 degrees C.

22. A processed ascidian as a processed product of a Halocynthia roretzi or another ascidian without encystment, temporal stability of contained plasmalogen being improved by freeze drying.

23. The processed ascidian according to the above 21, wherein a substance other than water other than the Halocynthia roretzi or another ascidian is not substantially contained.

24. The processed ascidian according to the above 21 or 22, wherein a plasmalogen content in a predetermined mass after freeze drying is 30% or more of the plasmalogen content in the predetermined mass immediately after freeze drying after a lapse of two months in an environment of 25 degrees C.

25. The processed ascidian according to the above 21 or 22, wherein a plasmalogen content in a predetermined mass after freeze drying is 10% or more, or, 30% or more of the plasmalogen content in the predetermined mass immediately after freeze drying after a lapse of two months in an environment of 35 degrees C.

26. The processed ascidian according to the above 21 or 22, wherein a plasmalogen content in a predetermined mass after freeze drying is 20% or more of the plasmalogen content in the predetermined mass immediately after freeze drying after a lapse of six months in an environment of 25 degrees C.

27. The processed ascidian according to the above 21 or 22, wherein a plasmalogen content in a predetermined mass after freeze drying is 10% or more, or, 30% or more of the plasmalogen content in the predetermined mass immediately after freeze drying after a lapse of six months in an environment of 35 degrees C.

28. The processed ascidian according to the above 21 or 22, wherein two months or more elapse at room temperature after freeze drying, and a plasmalogen content in a predetermined mass is 10% or more, 30% or more, or, 50% or more of the plasmalogen content in the predetermined mass immediately after freeze drying.

29. The processed ascidian according to the above 21 or 22, wherein six months or more elapse at room temperature after freeze drying, and a plasmalogen content in a predetermined mass is 10% or more, 20% or more, or, 40% or more of the plasmalogen content in the predetermined mass immediately after freeze drying.

30. A processed ascidian obtained by freeze-drying a Halocynthia roretzi or another ascidian without encystment, the processed ascidian being used for using plasmalogen contained after storage at room temperature to 35 degrees C. for two months or more after the freeze drying.

31. A processed ascidian obtained by freeze-drying a Halocynthia roretzi or another ascidian without encystment, the processed ascidian being used for using plasmalogen contained after storage at room temperature to 35 degrees C. for four months or more after the freeze drying.

(1) The method of using a plasmalogen contained in a processed ascidian of the present invention comprises the steps of: freeze-drying a Halocynthia roretzi or another ascidian without encystment (for example, all or a portion of a molluscous part of the Halocynthia roretzi or another ascidian or an edible portion of the Halocynthia roretzi or another ascidian) to obtain a processed ascidian; storing the processed ascidian at room temperature to 35 degrees C. for two months or more after the freeze drying; and using the processed ascidian stored for two months or more as described above to use a plasmalogen contained in the processed ascidian.

This method can comprise a step of subjecting the processed ascidian to use of a plasmalogen contained in the processed ascidian. In this case, for example, a person who uses the plasmalogen contained in the processed ascidian stored for two months or more as described above can use the processed ascidian to use the plasmalogen.

The freeze-drying step includes a step of preliminarily freezing all or a part of the Halocynthia roretzi or another ascidian without encystment, and a step of sublimation drying the preliminarily frozen Halocynthia roretzi or another ascidian by reducing the pressure. In addition, for example, the freeze-drying step can further include a step of drying so that there is no residual moisture in the Halocynthia roretzi or another ascidian.

A substance other than water may not be substantially added to the Halocynthia roretzi or another ascidian. In addition, extraction with an organic solvent may not be performed.

(2) The production method of the present invention is a method of producing a processed ascidian having good temporal stability of contained plasmalogen (that is, for example, plasmalogen decomposition, leakage, and the like are less likely to occur), and a Halocynthia roretzi or another ascidian without encystment is freeze-dried. The step of performing freeze drying can be the same as the step in the method of using the plasmalogen contained in the processed ascidian of the present invention. A substance other than water may not be substantially added to the Halocynthia roretzi or another ascidian. In addition, extraction with an organic solvent may not be performed.

(3) The processed ascidian of the present invention is a processed product of all or a part of Halocynthia roretzi or another ascidian without encystment, and the temporal stability of the contained plasmalogen is improved by freeze drying. A processed ascidian for using plasmalogen contained in the present invention is a processed ascidian obtained by freeze-drying a Halocynthia roretzi or another ascidian without encystment and is stored at room temperature to 35 degrees C. for a predetermined period or more after the freeze drying. In any case, the step of performing freeze drying can be the same as the step in the method of using plasmalogen contained in the processed ascidian of the present invention. A substance other than water may not be substantially added to the Halocynthia roretzi or another ascidian. In addition, extraction with an organic solvent may not be performed.

(4) The plasmalogen contained in the processed ascidian obtained by the production method of the present invention and the processed ascidian of the present invention are 1-alkenyl-2-acyl-sn-glycero-3-phosphate homologues having a vinyl ether bond in the sn-1 (a) position of a glycerol molecule, and are alkenyl ether-containing phospholipids generally represented by the following formula [1].

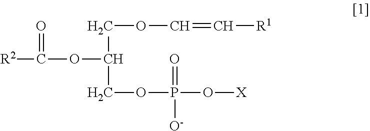

In the formula [1], R¹ is generally an alkyl group having 1 to 20 carbon atoms (for example, a dodecyl group, a tetradecyl group, a hexadecyl group, an octadecyl group, and an icosanyl group), R² is generally an aliphatic hydrocarbon group corresponding to a residue of a polyunsaturated fatty acid (including DHA, EPA, and arachidonic acid) or a monovalent unsaturated fatty acid such as oleic acid (examples of the R²CO-group include an octadecadienoyl group, an octadecatrienoyl group, an icosatetraenoyl group, an icosapentaenoyl group, a docosatetraenoyl group, a docosapentaenoyl group, and a docosahexaenoyl group. Preferred is an icosapentaenoyl group or a docosahexaenoyl group), and X is, for example, ethanolamine (the following formula [2]), choline (the following formula [3]), serine, inositol, or glycerol (preferred is ethanolamine or choline).

—CH₂CH₂N⁺H₃ [2]

—CH₂CH₂N⁺(CH₃)₃ [3]

(5) The processed ascidian can be produced, for example, as follows.

In the Halocynthia roretzi from which the encystment has been removed or another ascidian from which the encystment has been removed, all or a part (if it is frozen, it is preferably naturally thawed as much as possible) other than the encystment is treated (blanched) with hot water (for example, 90 to 95 degrees C. hot water). Preferably, the treatment is performed by immersion in hot water (for example, while stirring).

After treatment for a predetermined time (for example, 10 minutes), the ascidian from which the encystment has been removed is preliminarily frozen, thereafter sublimation dried by reducing the pressure in a drying chamber, and further dried so that no moisture remains, whereby a freeze-dried processed ascidian can be produced.

The obtained processed ascidian can be used through coarse pulverization, fine pulverization, sieving, and the like as necessary.

(6) The processed ascidian obtained by the production method of the present invention, the processed ascidian of the present invention, or the processed ascidian for using the plasmalogen contained according to the present invention can be, for example, used for human oral administration as a powder, a tablet, a hard capsule, or other forms, and can also be used for other uses. An oral dose of the processed ascidian obtained by the production method of the present invention or the processed ascidian of the present invention to human is preferably, for example, about 0.1 mg to 1000 mg/day in terms of the content of plasmalogen.

### EXAMPLES

### 1. Examples

(1) A molluscous part of the frozen ascidian (Halocynthia roretzi from which the encystment has been removed) (952 kg) was naturally thawed at room temperature and placed in a refrigerator for further thawing before complete thawing.

When 2.5 times of water in weight in proportion to the molluscous part of the ascidian thawed in the refrigerator was heated to 90 to 95 degrees C. in a tank, the thawed molluscous part of the ascidian was put into the water at a water temperature of about 70 degrees C.

Thereafter, the heating was continued, the mixture was stirred at a water temperature of 90 to 95 degrees C. for 10 minutes to treat (blanch) the molluscous part of the ascidian, and then the molluscous part of the ascidian was discharged from the tank together with hot water, and taken out.

The molluscous part of the ascidian (343.8 kg) taken out was packed in a freeze-drying tray and preliminarily frozen at −40 degrees C. for 12 hours or more. The inside of the drying chamber containing the molluscous part of the ascidian preliminarily frozen in a freeze dryer was reduced in pressure to 4 to 8 Pa, to sublimation dry the molluscous part of the ascidian for 65 hours or more. And the molluscous part of the ascidian was further dried so that no moisture remained, thereby obtaining a freeze-dried processed ascidian (113.8 kg) at a temperature of 60 degrees C.

The obtained processed ascidian was coarsely pulverized (about 1700 rpm) by a pulverizer of an 8 q (8 mm diameter) screen, then finely pulverized (8000 rpm) by a fine pulverizer of a 1.5 q (1.5 mm diameter) screen, and thereafter sieved with 32 mesh to obtain a finely pulverized product (113.1 kg) of the processed ascidian. A foreign matter may be removed by a foreign matter removing device using magnetic force and the like as necessary.

(2) When the content of plasmalogen in 100 g of the finely pulverized product of the obtained processed ascidian was measured, the content was 1129 mg. The content of plasmalogen was measured by an HPLC-ESI-MS/MS method. The same applies to the following examples.

(a) The finely pulverized product of the processed ascidian was stored in an environment (without humidity control) of 35 degrees C., and when two months, four months, and six months have elapsed, the content of plasmalogen in 100 g of the finely pulverized product of the processed ascidian was measured. As a result, the respective contents were 832 mg (73.7%) for two months, 632 mg (56.0%) for four months, and 663 mg (58.7%) for six months. Abnormalities in appearance, powder properties and odor were not observed. Bracketed numbering indicates a ratio to the content of plasmalogen in 100 g before storage, 1129 mg.

(b) When the finely pulverized product of the processed ascidian was stored at room temperature, and the content of plasmalogen in 100 g of the finely pulverized product of the processed ascidian was measured when six months have elapsed, the content was 788 mg (69.8%). Abnormalities in appearance, powder properties and odor were not observed. Bracketed numbering indicates a ratio to the content of plasmalogen in 100 g before storage, 1129 mg.

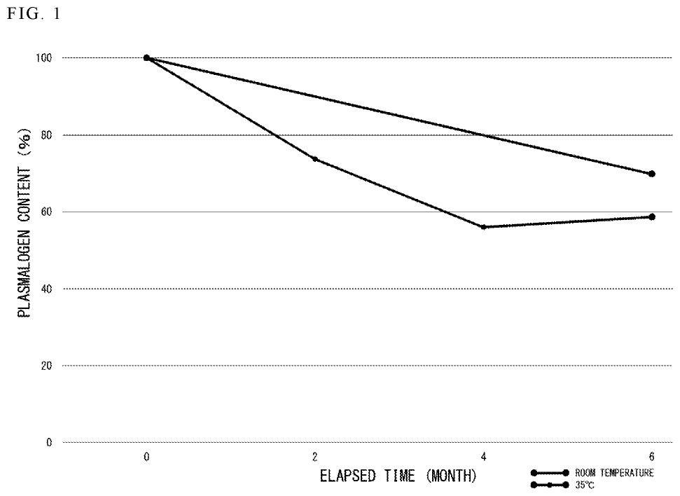

A temporal change of the content of the plasmalogen in 100 g of the processed ascidian is shown in FIG. 1 with the percentage with respect to the content before storage as the vertical axis and the elapsed month as the horizontal axis.

The higher the storage temperature (for example, at a temperature around room temperature or at a temperature other than the room temperature), the higher a rate at which the plasmalogen content in the processed ascidian having a predetermined mass decreases over time.

According to the measurement results and FIG. 1, it can be said that the plasmalogen content in the finely pulverized product of the processed ascidian having a predetermined mass after freeze-drying is a ratio as follows with respect to the plasmalogen content immediately after freeze-drying according to elapsed months.

When two months have elapsed:
it can be said that the plasmalogen content is 5 to 74% (for example, 5% or more, 10% or more, 20% or more, 30% or more, 40% or more, 50% or more, 60% or more, or 70% or more) in storage in an environment at 35 degrees C. (without humidity control), and 5 to 90% (for example, 5% or more, 10% or more, 20% or more, 30% or more, 40% or more, 50% or more, 60% or more, 70% or more, 80% or more, or 85% or more) at room temperature storage.

When four months have elapsed:
it can be said that the plasmalogen content is 5 to 56% (for example, 5% or more, 10% or more, 20% or more, 30% or more, 40% or more, or 50% or more) in storage in an environment at 35 degrees C. (without humidity control), and 5 to 80% (for example, 5% or more, 10% or more, 20% or more, 30% or more, 40% or more, 50% or more, 60% or more, 70% or more, or 75% or more) at room temperature storage.

When six months have elapsed:
it can be said that the plasmalogen content is 5 to 59% (for example, 5% or more, 10% or more, 20% or more, 30% or more, 40% or more, 50% or more, or 55% or more) in storage in an environment at 35 degrees C. (without humidity control), and 5 to 70% (for example, 5% or more, 10% or more, 20% or more, 30% or more, 40% or more, 50% or more, 60% or more, or 65% or more) at room temperature storage.

### 2. Comparative Examples

(1) According to the method described in Example 1 of JP 2019-162042 A, “50.8 g of a freeze-dried powder of an edible portion of ascidian was weighed, 66 mL of hexane, 121 mL of ethanol, and 11 mL of water were charged, mixed, and extracted with stirring at normal temperature for 30 minutes, and then filtered with qualitative filter paper (type: No. 2), an extraction liquid and a residue were separated, to the residue, 66 mL of hexane, 121 mL of ethanol, and 11 mL of water were charged again, mixed, and extracted with stirring at normal temperature for 30 minutes, and then filtered with the qualitative filter paper (type: No. 2), and the extraction liquid and the residue were separated. Solvent elimination from the combination of two times of the extraction liquid was conducted by vacuum drying to obtain an ascidian extract”, 0.9446 g of an ascidian extract powder was obtained.

(2) When the content of plasmalogen in the obtained ascidian extract powder was measured, the content was 5540 mg with respect to 100 g of the ascidian extract powder.

(a) The ascidian extract powder was stored in an environment of 40 degrees C., and at the time of elapse of 10 days, elapse of 20 days, elapse of one month, elapse of two months, and elapse of three months, the content of plasmalogen in the ascidian extract powder was measured, so that the respective contents were 2770 mg (50%) for 10 days, 831 mg (15%) for 20 days, 0 mg (0%) for one month, 0 mg (0%) for two months, and 0 mg (0%) for three months with respect to 100 g of the ascidian extract powder. Bracketed numbering indicates a ratio to the content of plasmalogen in 100 g before storage, 5540 mg.

(b) The ascidian extract powder was stored in an environment of 25 degrees C., and at the time of elapse of 10 days, elapse of 20 days, elapse of one month, elapse of two months, and elapse of three months, the content of plasmalogen in the ascidian extract powder was measured, so that the respective contents were 5097 mg (92%) for 10 days, 3712 mg (67%) for 20 days, 2936 mg (53%) for one month, 0 mg (0%) for two months, and 0 mg (0%) for three months with respect to 100 g of the ascidian extract powder. Bracketed numbering indicates a ratio to the content of plasmalogen in 100 g before storage, 5540 mg.

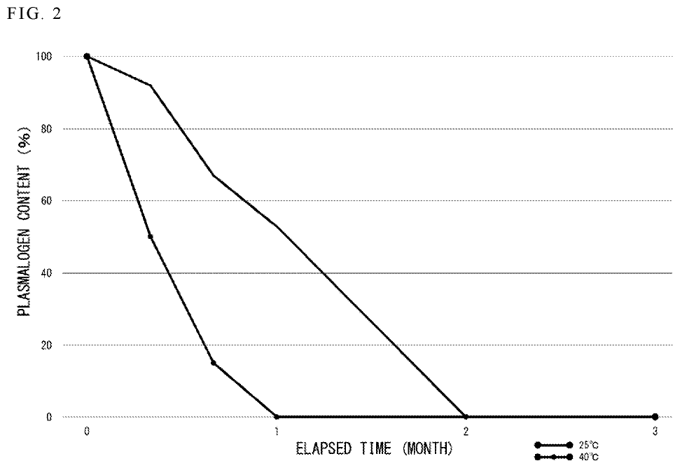

A temporal change of the content of the plasmalogen with respect to 100 g of the ascidian extract powder is shown in FIG. 2 with the percentage with respect to the content before storage as the vertical axis and the elapsed month as the horizontal axis.

What is claimed is:

1. A method of using a plasmalogen contained in a processed ascidian comprising the steps of: freeze-drying a Halocynthia roretzi or another ascidian without encystment to obtain a processed ascidian; storing the processed ascidian at room temperature to 35 degrees C. for two months or more after the freeze drying; and using the processed ascidian stored for two months or more as described above to use a plasmalogen contained in the processed ascidian.

2. The method according to claim 1, further comprising a step of subjecting the processed ascidian to use of a plasmalogen contained in the processed ascidian.

3. The method according to claim 1, further comprising a step of treating the Halocynthia roretzi or another ascidian without encystment with hot water before performing the freeze drying.

4. The method according to claim 1, wherein a plasmalogen content in a predetermined mass of the processed ascidian after freeze drying is 10% or more of the plasmalogen content in the predetermined mass immediately after freeze drying after a lapse of two months at room temperature.

5. The method according to claim 1, wherein a plasmalogen content in a predetermined mass of the processed ascidian after freeze drying is 30% or more of the plasmalogen content in the predetermined mass immediately after freeze drying after a lapse of two months at room temperature.

6. The method according to claim 1, wherein a plasmalogen content in a predetermined mass of the processed ascidian after freeze drying is 50% or more of the plasmalogen content in the predetermined mass immediately after freeze drying after a lapse of two months at room temperature.

7. The method according to claim 1, wherein a plasmalogen content in a predetermined mass of the processed ascidian after freeze drying is 30% or more of the plasmalogen content in the predetermined mass immediately after freeze drying after a lapse of two months in an environment of 25 degrees C.

8. The method according to claim 1, wherein a plasmalogen content in a predetermined mass of the processed ascidian after freeze drying is 10% or more of the plasmalogen content in the predetermined mass immediately after freeze drying after a lapse of two months in an environment of 35 degrees C.

9. The method according to claim 1, wherein a plasmalogen content in a predetermined mass of the processed ascidian after freeze drying is 30% or more of the plasmalogen content in the predetermined mass immediately after freeze drying after a lapse of two months in an environment of 35 degrees C.

10. A method of using a plasmalogen contained in a processed ascidian comprising the steps of: freeze-drying a Halocynthia roretzi or another ascidian without encystment to obtain a processed ascidian; storing the processed ascidian at room temperature to 35 degrees C. for six months or more after the freeze drying; and using the processed ascidian stored for four months or more as described above to use a plasmalogen contained in the processed ascidian.

11. The method according to claim 10, further comprising a step of subjecting the processed ascidian to use of a plasmalogen contained in the processed ascidian.

12. The method according to claim 10, further comprising a step of treating the Halocynthia roretzi or another ascidian without encystment with hot water before performing the freeze drying.

13. The method according to claim 10, wherein a plasmalogen content in a predetermined mass of the processed ascidian after freeze drying is 10% or more of the plasmalogen content in the predetermined mass immediately after freeze drying after a lapse of six months at room temperature.

14. The method according to claim 10, wherein a plasmalogen content in a predetermined mass of the processed ascidian after freeze drying is 20% or more of the plasmalogen content in the predetermined mass immediately after freeze drying after a lapse of six months at room temperature.

15. The method according to claim 10, wherein a plasmalogen content in a predetermined mass of the processed ascidian after freeze drying is 40% or more of the plasmalogen content in the predetermined mass immediately after freeze drying after a lapse of six months at room temperature.

16. The method according to claim 10, wherein a plasmalogen content in a predetermined mass of the processed ascidian after freeze drying is 20% or more of the plasmalogen content in the predetermined mass immediately after freeze drying after a lapse of six months in an environment of 25 degrees C.

17. The method according to claim 10, wherein a plasmalogen content in a predetermined mass of the processed ascidian after freeze drying is 10% or more of the plasmalogen content in the predetermined mass immediately after freeze drying after a lapse of six months in an environment of 35 degrees C.

18. The method according to claim 10, wherein a plasmalogen content in a predetermined mass of the processed ascidian after freeze drying is 30% or more of the plasmalogen content in the predetermined mass immediately after freeze drying after a lapse of six months in an environment of 35 degrees C.
